(19) United States
(12) Patent Application Publication
(10) Pub. No.: US 2022/0133240 A1
(43) Pub. Date: May 5, 2022

(54) SYSTEM AND METHOD FOR REDUCTING OR ELIMINATING ARTIFACTS IN MAGNECTIC RESONANCE IMAGING

(71) Applicant: CHENGDU YIJIAN MEDICAL TECHNOLOGY CO., LTD, CHENGDU (CN)

(72) Inventors: RUIXING ZHU, Chengdu (CN); ZHIZUN ZHANG, Chengdu (CN); HANGXUAN LI, Chengdu (CN)

(21) Appl. No.: 17/513,897

(22) Filed: Oct. 29, 2021

Related U.S. Application Data
(60) Provisional application No. 63/107,459, filed on Oct. 30, 2020.

(51) Int. Cl. A61B 5/00; A61B 5/055; G01R 33/56

(52) U.S. Cl. CPC A61B 5/7203; A61B 5/055; G01R 33/56; A61B 5/0046; A61B 5/7267

(57) ABSTRACT

A computer-implemented method for reducing or eliminating artifacts in MRI, includes steps of: S1: acquiring a plurality of MR signals mixed with EMI with different weightings from a plurality of receiver elements of at least one array coil; S2: obtaining EMI eliminated MR signals for each receiver element based on the MR signals mixed with EMI obtained in step S1; and S3: obtaining MR image based on the EMI eliminated MR signals obtained in step S2.

### CROSS REFERENCE TO RELATED APPLICATIONS

This application claims priority to U.S. Provisional Application No. 63/107,459, entitled “Systems and Methods for Eliminating Electromagnetic interference for Magnetic Resonance Imaging” filed on Oct. 30, 2020, which is hereby incorporated by reference herein as if set forth in its entirety.

### TECHNICAL FIELD

The present disclosure relates to Magnetic Resonance Imaging, and particularly to a system and method for reducing or eliminating artifacts in Magnetic Resonance Imaging.

### BACKGROUND

Magnetic resonance imaging (MRI) has impacted modern healthcare tremendously and is recognized by clinicians as the most valuable medical device innovation in the last three decades. MRI is now a routine procedure in diagnosis and management of various diseases and injuries, and over 100 million MRI investigations are performed each year worldwide. It is the most powerful diagnostic imaging modality because of its capability in detecting and characterizing pathological tissues with high sensitivity and specificity in an inherently quantitative, non-invasive and non-ionizing manner.

Artifacts usually occur in MRI, which affects the image quality. Electromagnetic interference (EMI) is one of the reasons causing artifacts. Therefore, the conventional MRI scanners are extremely expensive to install and maintain due to extensive infrastructural requirements and modifications needed to site the scanners, including the dedicated radio-frequency shielding room preparations to eliminate external EMI during MRI data acquisition.

### SUMMARY

In view of this, there is a desire in the art to provide a new system and method for reducing or eliminating artifacts in magnetic resonance imaging.

In one aspect, the present disclosure provides a computer-implemented method for reducing or eliminating artifacts in MRI, including steps of:
S1: acquiring a plurality of MR signals mixed with EMI with different weightings from a plurality of receiver elements of at least one array coil;
S2: obtaining EMI eliminated MR signals for each receiver element based on the MR signals mixed with EMI obtained in step S1; and
S3: obtaining MR image based on the EMI eliminated MR signals obtained in step S2.

In another aspect, the present disclosure provides a computer-implemented method for reducing or eliminating artifacts in MRI, including steps of:
S1: acquiring a plurality of MR signals mixed with EMI with different weightings from a plurality of receiver elements of at least one array coil;
S25: designing and training a deep learning model to establish relationships between EMI signals sampled by different receiver elements within each of the at least one array coil in absence of any MR signal, from which EMI signals from a group of receiver elements are estimated from EMI signals in other receiver elements; and
S26: for each set of actual measurements obtained during a specific subject MRI scan, feeding the MR signals acquired in step S1 to the trained model to estimate the EMI in the MR signals and outputting reconstructed MR images.

In a third aspect, the present disclosure provides a system including:
at least on array coil having a plurality of receiver elements;
at least one computer hardware processor;
at least one non-transitory computer-readable storage medium; and
at least one computer program stored in the at least one non-transitory computer-readable storage medium and executable on the at least one computer hardware processor,
wherein when executing the at least one computer program, the at least one computer hardware processor implements the above methods.

### BRIEF DESCRIPTION OF THE DRAWINGS

To describe the technical schemes in the embodiments of the present disclosure or in the prior art more clearly, the following briefly introduces the drawings required for describing the embodiments or the prior art. It should be understood that, the drawings in the following description merely show sonic embodiments of the present disclosure. For those skilled in the art, other drawings can be obtained according to the drawings without creative efforts.

FIG. 1 is a flow chart of a method for reducing or eliminating artifacts in Magnetic Resonance Imaging according to an embodiment of the present disclosure.

FIG. 2 is a flow chart of a method for reducing or eliminating artifacts in Magnetic Resonance Imaging according to another embodiment of the present disclosure.

FIG. 3 is a schematic block diagram of a system for reducing or eliminating artifacts in Magnetic Resonance Imaging according to an embodiment of the present disclosure.

FIG. 4Ashows an image of clean MR signal.

FIG. 4B shows an image of EMI.

FIG. 4C shows an image of noise.

FIG. 4D shows an image of MR signal contaminated with EMI and noise.

FIG. 5A illustrates the k-space of clean MR signal corresponding to that shown in FIG. 4A.

FIG. 5B illustrates the k-space of EMI corresponding to that shown in FIG. 4B.

FIG. 5C illustrates the k-space of noise corresponding to that shown in FIG. 4C.

FIG. 5D illustrates the k-space of MR signal contaminated with EMI and noise corresponding to that shown in FIG. 4D.

FIG. 6 illustrates workflow for obtaining an image by the method according to one embodiment of the present invention using deep learning.

FIG. 7 illustrates workflow for obtaining an image by the method according to another embodiment of the present invention using deep learning.

FIG. 8A shows a k-space of MRI with EMI.

FIG. 8B shows the MR image corresponding to FIG. 8A.

FIG. 8C shows the corresponding k-space corrected by the method according to one embodiment of the present disclosure.

FIG. 8D show the corresponding final MR image corrected by the according to one embodiment of the present disclosure.

FIG. 9A shows human brain MR images contaminated with EMI and noise.

FIG. 9B shows human brain images obtained by the method according to one embodiment of the present disclosure.

### DETAILED DESCRIPTION

In the following descriptions, for purposes of explanation instead of limitation, specific details such as particular system architecture and technique are set forth in order to provide a thorough understanding of embodiments of the present disclosure. However, it will be apparent to those skilled in the art that the present disclosure may be implemented in other embodiments that are less specific of these details. In other instances, detailed descriptions of well-known systems, devices, circuits, and methods are omitted so as not to obscure the description of the present disclosure with unnecessary detail.

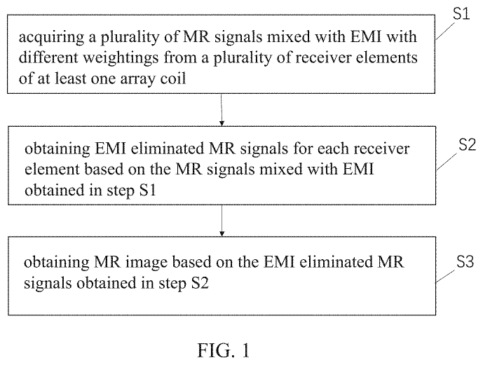

FIG. 1 shows a computer-implemented method for reducing or eliminating artifacts in MRI according to an embodiment of the present disclosure. The method includes the steps as follows.

S1: Acquiring a plurality of MR signals mixed with EMI with different weightings from a plurality of receiver elements of at least one array coil;

S2: obtaining EMI eliminated MR signal for each receiver element based on the MR signals mixed with EMI obtained in step S1; and

S3: obtaining MR image based on the EMI eliminated MR signals obtained in step S2.

In some embodiment, the method according to the present disclosure further includes a step S4 of optimizing certain receiver elements to mainly detect EMI signals from broad range of sources with high sensitivity along three different polarization directions through coil designs and their spatial distribution.

Specifically, in step S1, the signals can be acquired from the specific subject, a group of subjects, phantoms, or in absence of any subject or phantom with and without transmitting excitation RF. Data measurements can also be acquired directly and simultaneously during the specific patient MRI scan, where EMI signals can be acquired within each NMR excitation repetition period at times where MR signals are expected to be zero.

Preferably, the receiver elements are arranged at different locations and orientations inside and/or near the MR scanner and subjected to simultaneously sense EMI during MRI scan. Preferably, at least one coil of each of the at least one array coil has different receiving RF coil design, RF polarization orientation, location and/or different sensitivity to EMI and MR signals (including zero or maximum sensitivity to EMI or MR signal).

The coils may be rigid or semi-rigid. The coil may be small and flexible surface-like RF coils that can be easily amounted on the subject/patient skin to directly detect various EMI signals picked up by subject/patient body.

MR signals can be spatially-encoded by the magnetic gradient coils, and the received MR signal is modulated by the coil sensitivity maps.

EMI can originate from many sources, both external or internal, including power lines, elevators, etc. EMI is independent from the MRI encoding gradients. It is noted that the detected EMI can also be related to the sequences and parameters that are used to collect data. EMI can depend on many parameters, including the main magnetic field (B₀), bandwidth (BW), and can also change after a long scan.

The features of detected EMI are related to the locations, orientations, and physical characteristics of the RF coils (i.e., RF coil design) with respect to EMI source, and characteristics of the subject to be imaged.

For far field sources, it is preferred to arrange multiple receiver elements of each array coil that have different orientations (e.g., X, Y, and Z) around the subject. This is to characterize the EMI of different polarities.

For near field sources, it is preferred to use multiple array coils to map its spatial distribution. For example, the human body can act as an antenna which can pick up and emit EMI, which can further complicate the situation. In other words, human body is a conductive structure, and other parts of the body outside the magnet will couple with the environment. Therefore, using multiple coils to detect EMI distribution at different locations and orientations can further improve the MR scans.

At ultra-low magnetic field B₀ (0.001-0.1 Tesla, T), the wavelength of MR signal is sufficiently long. This means that the EMI which can interact with MRI acquisition also has long wavelength. Therefore, the number of sensors i.e., array coils, required to characterize EMI can be small. On the other hand, the wavelength is short at mid-field to high-field magnetic field B₀ and more array coils will be needed. At high field, the noise is dominated by the thermal noise from the sample, while at low field, the noise is dominated by the coil and receive chain electrical noise.

Preferably, to precisely characterize EMI, multiple array coils are placed surrounding the subject with different orientations (at least 3 orientations, e.g., X, Y, and Z).

In some embodiment, Step S2 may be implemented by means of blind source separation and/or deep learning model.

In particular, the MR signal detection by the receiver elements can be modeled as below, i.e., the MR images are modulated by the coil sensitivity maps (CSMs) and then Fourier transformed to k-space. The modulation by the CSMs in image space is equivalent to convolution in k-space. Therefore, the MR signal detected from each receiver element cannot be simply treated as from the same source. Instead, they should be treated as from independent sources, unless some of the receiver elements have the same coil sensitivity maps (or multiplied by a constant complex number). This happens when the receiver elements are far from the subject.

The EMI can be considered to be statistically invariant across the whole k-space, and EMI can be treated as from the same source if the EMI source is far from all receiver elements.

As MR signal and EMI are statistically independent of each other, the EMI elimination can be modeled as a blind source separation problem. Step S2 may include following steps:

S21: estimating mixing/unmixing matrix based on the MR signals mixed with EMI;

S22: identifying EMI and noise from unmixed sources;

S23: setting the weightings for EMI and noise related sources to zero; and

S24: obtaining EMI eliminated MR signal by re-mixing the sources using estimated mixing matrix.

As for blind source separation, the number of measurements should be larger than the number of sources. As such, at least two receiver elements distant from the imaging object are provided in the method.

Specifically, the signal detection can be modeled as,

m=Ws

where m is the N measurements, s is the M sources, W is an N×M mixing matrix which linearly combines the sources.

When N>M, ICA (independent component analysis) can be used to determine the W and s from the measurements m, wherein s contains at most M−1 sources related to MR or EMI signals and 1 source related to thermal noise.

In some embodiments, the mixing matrix may be estimated using ICA, which can be implemented with various models, including fixed-point algorithm with projection pursuit, informax algorithm which finds independent signals by maximizing the entropy, etc.

In some embodiments, the coil sensitivity information can also be incorporated into ICA model.

In some embodiments, the mixing matrix may be estimated using PCA (Principal component analysis). PCA can be done by eigenvalue decomposition of a data covariance (or correlation) matrix or singular value decomposition (SVD) of a data matrix.

Specifically, the k-space (or image space) of MR data acquired with multiple receiver elements can be reshaped into a matrix or tensor, and decomposed using SVD.

Particularly, the unmixed sources can be identified through a variety of methods. In some embodiments, EMI and noise can be characterized through spectrum analysis. For example, as the power spectrum distribution characteristics of MR signal and EMI signal are different, the non-parametric classical spectrum estimation can be carried out by Welch method, by dividing the signal into partially overlapping data segments, respectively adding windows to calculate the periodogram, and finally taking the average to complete the analysis.

In some embodiments, an autoregressive moving average (ARMA) model can also be used for EMI and noise analysis. The use of ARMA model can improve the poor performance and low resolution of classical spectrum estimation. Assuming that the source signal s is the output of an input sequence passing through a linear time-invariant system H(z) with both zero and maximum, the parameters of H(z) are estimated by s or its autocorrelation function r, and then the power spectrum is estimated by H(z), specifically, the modified Yule-Walker method can be used, after the power spectral density of each source is obtained, it is classified. If the power spectral density of the source is higher than the preset threshold in the preset frequency band, the source is identified as electromagnetic interference EMI. The MR signal can be identified through peak detection, i.e., checking whether the peak value corresponding to k-space center is above than a threshold.

In some embodiments, the unmixed sources can be identified in image-space. The EMI typically presents as bright and noisy band(s) in image-space overlapping the background or object region.

In above method, it does not require training data to estimate the mixing matrix, however, in some embodiments, training data may also be incorporated to get a more robust and accurate estimation of the mixing matrix.

In some embodiment, with sufficient training data available, Step S2 may be implemented by means of deep learning model. For this purpose, a neural network architecture is designed, which inputs the EMI and/or noise contaminated MR signal, and outputs EMI and/or noise eliminated MR signals (also referred to as clean MR signals hereinafter). The model training can use simulated, and/or data from real subject/patients/phantoms that are acquired during, before, or/and after specific patient scan.

In particular, the step S2 may include the following steps:

S25: designing and training a deep learning model to establish relationships between EMI signals sampled by different receiver elements within each array coil in absence of any MR signal, from which EMI signals from a group of receiver elements can be estimated from EMI signals in other receiver elements;

S26: for each set of actual measurements obtained from during a specific subject/patient MRI scan, the acquired MR signals will be fed to the trained models above, the contaminated EMI signals will be estimated and removed, yielding EMI free MR signal for the final MRI image reconstruction.

The neural network architecture may be artificial neural network (ANN), convolutional neural network (CNN), generative adversarial network (GAN), and etc.

Take the convolutional neural network as an example. The input of the convolutional neural network is a complex image data, for example, a complex image data with a size of 256×256×2×2, wherein the length of the penultimate dimension is 2 to represent two receivers, and the length of the last dimension is 2 to represent the real part channel and the imaginary part channel respectively. The output of the convolutional neural network model is 256×256×2×2 residual image data, corresponding to the real and imaginary parts of the residual image data of the two receivers. The convolutional neural network model includes five convolutional layers and activation functions, namely, 9×9 convolutional layer, ReLu activation layer, 7×7 convolutional layer, ReLu activation layer; 5×5 convolutional layer, ReLu activation layer, 5×5 convolutional layer, ReLu activation layer, 3×3 convolutional layer, and the corresponding output channels of each convolutional layer are 128, 64, 32, 32, and 4, respectively. When training the model, the ADAM or SGD optimizer can be used to minimize the loss function, and choose Mean Squared Error (MSE) for the loss function.

In some embodiment, the output of the neural network architecture may be clean MR signals, which are then connected to another network configured for subsequent image reconstruction (e.g., parallel imaging reconstruction, coil compression). That is, the another network inputs the clean MR signals and output MR images.

In some embodiments, it is possible to design a neural network that does not require subsequent image reconstruction process, that is, using a single neural network directly outputs the reconstructed image.

In addition, the EMI elimination process can occur in the mixed space between the original data k-space and the image space, such as kx-y space, x-ky space, kx-y-z space and kx-ky-z, etc.

In some embodiment, the neural network can also be designed to output the EMI, and clean MR signal can be obtained by subtracting the EMI output from the contaminated MR signal. This way is especially useful in these cases where training data is available.

In some embodiments, the clean (EMI-free) MR data may be acquired from a well-shielded MR system for training data.

In some embodiments, the clean MR data may be obtained with simulation. MRI data may be generated by modulating a complex image with coil sensitivities of maps of the array coils, and then transformed into Fourier domain (frequency/measurement space).

In some embodiments, the EMI may be obtained with simulation. EMI can be generated by randomizing the phase, central frequency, and bandwidth of the EMI. Additive white Gaussian noise may also be added to mimic the real situation.

In some embodiments, the EMI may be acquired by turning off the MR excitation RF transmission or removing any MR signal sources (such as human body parts or phantoms) from MR RF coils. As is noted that detected EMI can also be related to the sequences and parameters (especially bandwidth) that are used to collect data. For different sequences and different parameter settings, EMI may need to be acquired with matched parameters.

In some embodiments, the EMI training data can be acquired simultaneously during the patient MRI scan, where EMI signals can be acquired within each NMR excitation repetition period at time where MR signals are expected to be zero.

In some embodiments, the MR data contaminated with EMI may be obtained by adding the simulated or acquired EMI/noise to clean MR signal for machine learning.

In some embodiments with multiple array coils, the positioning (locations and orientations) of the array coils can be automatically optimized.

According to the method of the present disclosure, after acquiring the MR signals mixed with EMI from the array coils, subsequent reduction and/or elimination of EMI from the MR signals can be achieved through post-processing and use of analytical and computational approaches, including blind separation and the deep learning techniques to recognize or even predict systematic EMI patterns for subsequent reduction and/or elimination. By means of the method according to the present disclosure, EMI is minimized during image acquisition and reconstruction without sacrificing subject comfort and operating ease.

A further method for reducing or eliminating EMI from MR signal according to another embodiment of the present disclosure is provided. The method includes the following steps:

S1a: obtaining calibration data from the specific subject, a group of subjects, phantoms, or in absence of any subject or phantom with and without transmission excitation RF;

S2a: using calibration data to establish the spatiotemporal multi-dimensional coupling linear matrices between receiver elements, which can be used to extract EMI of each receiver element from multiple measurements;

Step S3a: using the estimated linear coupling matrix operations to extract EMI from EMI contaminated MR signals; and

Step S4a: obtaining clean MR signals by subtracting the extracted EMI.

Preferably, certain receiver elements and locations can be optimized to mainly detect EMI signals from broad range of sources with sensitivity along 3 different polarization directions through coil designs and their optimal spatial distribution.

In particular, in Step 1a, the calibration data can also be acquired simultaneously during the specific patient MRI scan, where EMI signals can be acquired within each NMR excitation repetition period at times where MR signals are expected to be zero.

Assuming that there are C receiver elements, the 1st to Kth receiver elements are adjusted to a certain degree of sensitivity to magnetic resonance MR and electromagnetic interference signals EMI signals. For the pair of K+1 to Cth receiver elements, the sensitivity of the MR signal is adjusted to be close to 0, and the sensitivity to the EMI is adjusted to the maximum, that is, only the EMI is received. For each receiver element i, the collected training data containing only the EMI is nᵢ, where i=1, 2, 3, . . . , C. Using the data of the last C−K receiver components to represent the first K receivers, the following formula can be established:

[n_{K+1}, n_{K+2}, . . . , n_{C}][w_{K+1}, w_{K+2}, . . . , w_{C}]^{T}=[n₁, n₂, . . . , n_{K}]

which can be simplified as:

N_{EMI}W=Nₘᵢₓ

wherein the sizes of the N_{EMI}, W, and Nₘᵢₓ matrices are R×(C−K), (C−K)×K, R×K, and R is the number of repeated measurements.

It can be concluded that: W=N_{EMI}⁺Nₘᵢₓ, where + represents Moore-Penrose pseudo-inverse.

Assuming that the data collected by the first K and the next C−K receivers during the actual scan are Xₘᵢₓ and X_{EM}, the data Y_{MR} after the first K receiver elements eliminate the electromagnetic interference signal EMI signal can be expressed as:

Y_{MR}=Xₘᵢₓ−X_{EMI}W

In some embodiments, the data of the K receivers for MR signals can be combined by means of RSOS (Root Sum Of Squares) after image reconstruction, or through Singular Value Decomposition (SVD) before image reconstruction.

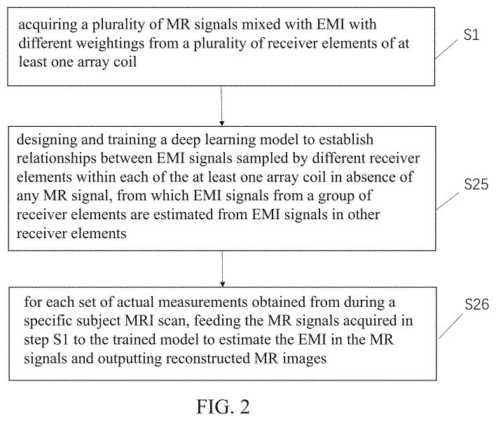

FIG. 2 is a schematic block diagram of a system for reducing or eliminating artifacts in MRI according to an embodiment of the present disclosure. The system corresponds to the computer-implemented methods for reducing or eliminating artifacts in MRI described in the above embodiments.

As shown in FIG. 2, the system includes at least one array coil 10 having multiple receiver elements 12 arranged at different locations and orientations inside and near the MRI scanner, and an analyzing apparatus which includes at least one computer hardware processor 20, at least one non-transitory computer-readable storage medium 30, and at least one computer program 40 stored in the at least one non-transitory computer-readable storage medium 30 and executable on the at least one computer hardware processor 20. When executing (instructions in) the at least one computer program 40, the at least one computer hardware processor 20 implements the method for reducing or eliminating artifacts in MRI described in the above embodiments shown in FIG. 1.

Exemplarily, the at least one computer program 40 may be divided into one or more modules, and the one or more modules are stored in the at least one non-transitory computer-readable storage medium 30 and executed by the at least one computer hardware processor 20 to realize the present disclosure. The one or more modules may be a series of computer program instruction sections capable of performing a specific function, and the instruction sections are for describing the execution process of the at least one computer program 10 in the system.

In this embodiment, the at least one computer program 10 includes an acquisition module 42, and a separation module 44. The acquisition module 42 is configured to acquire MR signals mixed with EMI with different weightings from at least two receiver elements of the at least one array coil 10. The separation module 44 is configured to separate the MR signal and the EMI signal of the outcome of the acquisition module 42 to obtain EMI eliminated MR signal.

For the details, references are made to the above descriptions discussed in the methods, which will not be repeated here.

The method and system for reducing or eliminating artifacts in MRI can be applied to any ultra-low field (0.001 T to 0.1 T), low-field (0.1 T to 0.5 T), mid-field (0.5 T to 3 T), and high-field (3 T and above) MRI systems that are equipped with multiple receiver element RF electronics. Multiple receiver element RF electronics are standard in virtually all clinical and non-clinical MRI systems.

The method and system are proposed for MR scan in MR systems without RF shielding, but it can also be applied to MR scan in MR systems with RF shielding when the shielding performance is limited.

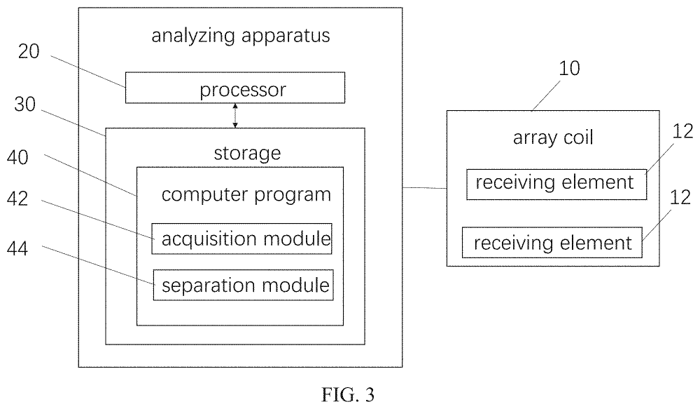

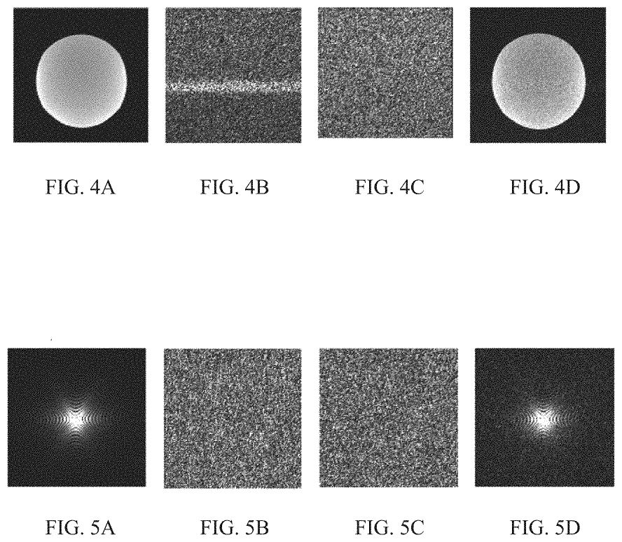

FIG. 4A shows an image of clean MR signal. FIG. 4B shows an image of EMI. FIG. 4C shows an image of noise. FIG. 4D shows an image of MR signal contaminated with EMI and noise. FIG. 5A illustrates the k-space of clean MR signal corresponding to that shown in FIG. 4A, FIG. 5B illustrates the k-space of EMI corresponding to that shown in FIG. 4B, FIG. 5C illustrates the k-space of noise corresponding to that shown in FIG. 4C. FIG. 5D illustrates the k-space of MR signal contaminated with EMI and noise corresponding to that shown in FIG. 4D. As shown in FIGS. 4A to 4D and FIGS. 5A to 3D, when the EMI and/or the noise signal involved, the images in the image space and the images in the k-space are affected.

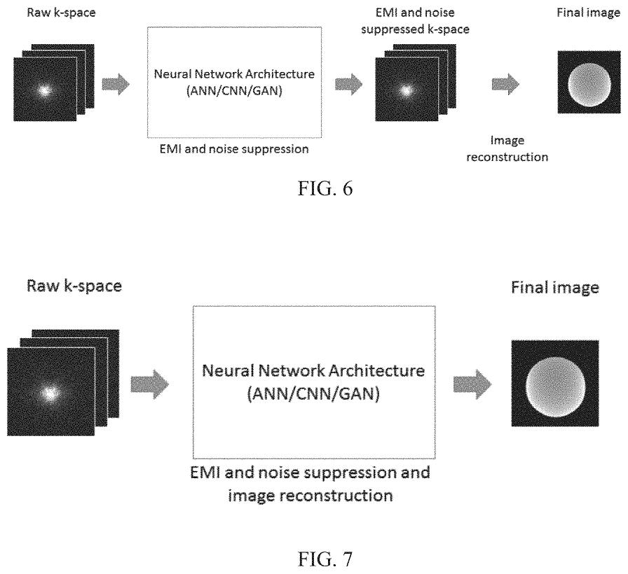

FIG. 6 illustrates workflow for obtaining an image by the method according to another embodiment of the present invention using deep learning. FIG. 6 illustrates that the image obtained by the method after the EMI elimination has a higher signal-to-noise ratio, and the image is also clearer. As shown in FIG. 6, the neural network architecture outputs the EMI and noise eliminated MR signals, which is used for the subsequent image reconstruction to obtain final image.

FIG. 7 illustrates workflow for obtaining an image by the method according to another embodiment of the present invention using deep learning. FIG. 7 illustrates that the image obtained by the method after the EMI elimination has a higher signal-to-noise ratio, and the image is also clearer. As shown in FIG. 7, the neural network architecture directly outputs the MR image without additional reconstruction process.

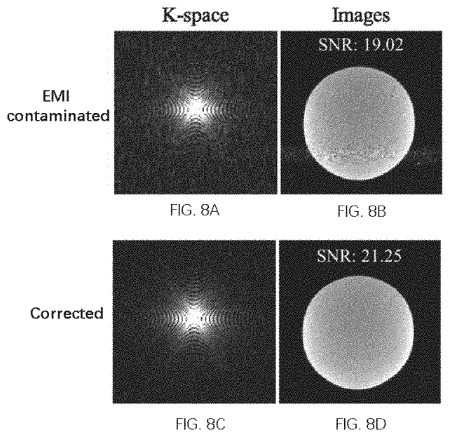

FIG. 8A shows a k-space of MRI with EMI. FIG. 8B shows the MR image corresponding to FIG. 8A. FIG. 8C shows the corresponding k-space corrected by the method according to one embodiment of the present disclosure. FIG. 8D shows the corresponding final MR image corrected by the according to one embodiment of the present disclosure. Through the comparison, it can be concluded that the images obtained after the EMI elimination using the method have higher signal-to-noise ratios, and the images are also clearer.

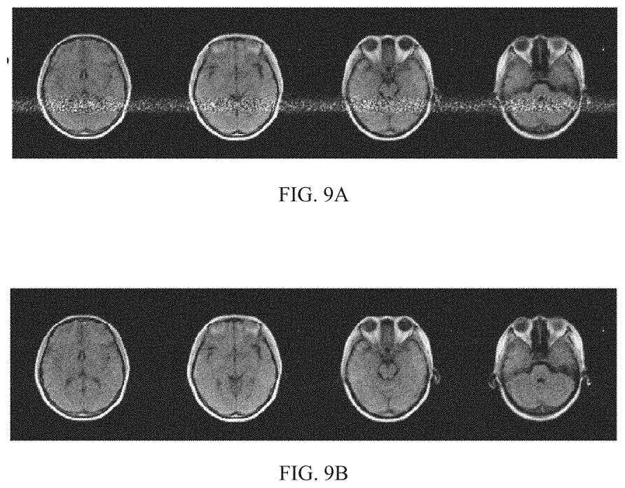

FIG. 9A shows human brain MR images contaminated with EMI and noise. FIG. 9B shows human brain images obtained by the method according to one embodiment of the present disclosure. Through the comparison, it can be concluded that the image obtained after the electromagnetic interference signal EMI is eliminated by using the artifact elimination method is clearer, and the noise caused by the electromagnetic interference signal EMI can be removed.

In the embodiments provided by the present disclosure, it should be understood that the disclosed method, system, and apparatus (or device) may be implemented in other manners. For example, the above-mentioned system and apparatus embodiment is merely exemplary. For example, the division of modules or units is merely a logical functional division, and other division manner may be used in actual implementations, that is, multiple units or components may be combined or be integrated into another system, or some of the features may be ignored or not performed.

The units described as separate components may or may not be physically separated. The components represented as units may or may not be physical units, that is, may be located in one place or be distributed to multiple network units. Some or all of the units may be selected according to actual needs to achieve the objectives of this embodiment.

In addition, each of the functional units or modules in each of the embodiments of the present disclosure can be integrated in one processing unit. Each unit or modules can physically exist alone, or two or more units can be integrated in one unit, or two or more modules can be integrated in one module. The above-mentioned integrated unit or module can be implemented either in the form of hardware, or in the form of software functional units or modules.

The integrated unit or module can be stored in a computer-readable storage medium if it is implemented in the form of a software functional unit and sold or utilized as a separate product. Based on this understanding, the technical solution of the present disclosure, either essentially or in part, contributes to the prior art, or all or a part of the technical solution can be embodied in the form of a software product. The software product is stored in a storage medium, which includes a number of instructions for enabling a computer device (which can be a personal computer, a server, a network device, etc.) or a processor to execute all or a part of the steps of the methods described in each of the embodiments of the present disclosure. The above-mentioned storage medium includes a variety of media such as a USB disk, a mobile hard disk, a read-only memory (ROM), a random access memory (RAM), a magnetic disk, and an optical disk which is capable of storing program codes.

As mentioned above, the forgoing embodiments are merely intended for describing but not for limiting the technical schemes of the present disclosure. Although the present disclosure is described in detail with reference to the above-mentioned embodiments, it should be understood by those skilled in the art that, the technical schemes in each of the above-mentioned embodiments may still be modified, or some of the technical features may be equivalently replaced, while these modifications or replacements do not make the essence of the corresponding technical schemes depart from the spirit and scope of the technical schemes of each of the embodiments of the present disclosure, and should be included within the scope of the present disclosure.

What is claimed is:

1. A computer-implemented method for reducing or eliminating artifacts in MRI, comprising the steps of:
S1: acquiring a plurality of MR signals mixed with EMI with different weightings from a plurality of receiver elements of at least one array coil;
S2: obtaining EMI eliminated MR signals for each receiver element based on the MR signals mixed with EMI obtained in step S1; and
S3: obtaining MR image based on the EMI eliminated MR signals obtained in step S2.

2. The method of claim 1, further comprises a step of:
optimizing at least one of the receiver elements to mainly detect EMI signals from broad range of sources with high sensitivity along three different polarization directions through coil designs and their spatial distribution.

3. The method of claim 1, wherein the receiver elements are arranged at different locations and/or orientations.

4. The method of claim 1, wherein at least one coil of the at least one array coil has different receiving RF coil design, RF polarization orientation, location and/or different sensitivity to EMI and MR signal.

5. The method of claim 1, wherein the at least one array coil comprises a plurality of array coils arranged at different locations and/or orientations.

6. The method of claim 1, wherein the step S2 is implemented with blind source separation.

7. The method of claim 6, wherein the step S2 comprises the steps of:
S21: estimating mixing/unmixing matrix based on the MR signals mixed with EMI;
S22: identifying EMI and noise from unmixed sources;
S23: setting the weightings for EMI and noise related sources to zero; and
S24: obtaining EMI eliminated MR signal by re-mixing the sources using estimated mixing matrix.

8. The method of claim 7, wherein in step S21, independent component analysis is used to estimate the mixing matrix.

9. The method of claim 8, wherein the independent component analysis implemented with fixed-point algorithm with projection pursuit, or informax algorithm which finds independent signals by maximizing the entropy.

10. The method of claim 7, wherein in step S21, principal component analysis is used to estimate the mixing matrix.

11. The method of claim 10, wherein the principal component analysis is implemented by eigenvalue decomposition of a data covariance or correlation matrix, or singular value decomposition of a data matrix.

12. The method of claim 1, wherein the Step S2 is implemented by means of deep learning model.

13. The method of claim 1, wherein the Step S2 comprises steps of:
S25: designing and training a deep learning model to establish relationships between EMI signals sampled by different receiver elements within each of the at least one array coil in absence of any MR signal, from which EMI signals from a group of receiver elements are estimated from EMI signals in other receiver elements; and
S26: for each set of actual measurements obtained from during a specific subject MRI scan, feeding the MR signals acquired in step S1 to the trained model to estimate the EMI in the MR signals.

14. The method of claim 12, wherein in step S2, a neural network architecture is designed, which inputs the MR signals mixed with EMI acquired in step S1, and outputs EMI eliminated MR signals.

15. The method of claim 12, wherein in step S2, a neural network architecture is designed, which inputs the MR signals mixed with EMI acquired in step S1, and outputs EMI, and wherein the step S2 further comprises a step of subtracting the EMI output from the MR signals mixed with EMI obtained in step S1.

16. The method of claim 1, further comprises a step of:
adjusting coil sensitivity to EMT of directions X, Y, and Z of the at least one array coil.

17. A computer-implemented method for reducing or eliminating artifacts in MRI, comprising steps of:
S1: acquiring a plurality of MR signals mixed with EMI with different weightings from a plurality of receiver elements of at least one array coil;
S25: designing and training a deep learning model to establish relationships between EMI signals sampled by different receiver elements within each of the at least one array coil in absence of any MR signal, from which EMI signals from a group of receiver elements are estimated from EMI signals in other receiver elements; and
S26: for each set of actual measurements obtained from during a specific subject MRI scan, feeding the MR signals acquired in step Si to the trained model to estimate the EMI in the MR signals and outputting reconstructed MR images.

18. The method of claim 1, wherein the deep learning model is designed as a neural network architecture, and the neural network architecture is an artificial neural network, a convolutional neural network, or a generative adversarial network.

19. A system for reducing or eliminating artifacts in MRI, comprising:
at least one array coil having a plurality of receiver elements;
at least one computer hardware processor;
at least one non-transitory computer-readable storage medium; and
at least one computer program stored in the at least one non-transitory computer-readable storage medium and executable on the at least one computer hardware processor,
wherein when executing the at least one computer program, the at least one computer hardware processor implements the method according to claim 1.

20. The system of claim 19, wherein the at least one computer program comprises:
acquisition module, configured to acquire MR signals mixed with EMI with different weightings from at least two receiver elements of the at least one array coil; and
separation module, configured to separate the MR signals and the EMI of the MR signals mixed with EMI obtained from the acquisition module.
